(19) United States
(12) Patent Application Publication
(10) Pub. No.: US 2026/0083978 A1
(43) Pub. Date: Mar. 26, 2026

(54) LIGHT RADIATING FIBER PROBE

(71) Applicant: FURUKAWA ELECTRIC CO., LTD., Tokyo (JP)

(72) Inventors: Hideaki HASEGAWA, Tokyo (JP); Keisuke TAKE, Tokyo (JP); Atsushi HIMURA, Tokyo (JP); Junichi HASEGAWA, Tokyo (JP)

(21) Appl. No.: 19/112,969

(22) PCT Filed: Aug. 31, 2023
(86) PCT No.: PCT/JP2023/031950

§ 371 Date: Mar. 19, 2025

(30) Foreign Application Priority Data
Sep. 21, 2022 (JP) 2022-150452

(51) Int. Cl. A61N 5/06

(52) U.S. Cl. CPC A61N 5/0603; A61N 2005/0609; A61N 2005/063; A61N 2005/0664

(57) ABSTRACT

The present invention provides a light radiating fiber probe that can emit light with high beam quality in a direction inclined relative to an axial direction of an optical transmission cable. A light radiating fiber probe 1 is mounted in medical equipment for treatment, and includes: an optical transmission cable 10 through which light emitted from a light source is transmitted; and a light refracting unit 20 provided on a leading end 13 side of the optical transmission cable 10 and refracting, via two or more refracting surfaces 21, laser light L emitted from the optical transmission cable 10 to emit light inclined by a predetermined angle or larger relative to an axial direction X of the optical transmission cable 10.

### TECHNICAL FIELD

The present invention relates to an optical irradiation fiber probe.

### BACKGROUND ART

Conventionally, in the medical field, optical irradiation fiber probes have been used for insertion into the human body and irradiating cells with light. For example, Patent Document 1 discloses a lateral emission device, in which a prism lens with a gold-coated inclined distal surface is arranged at one end of an optical fiber. An optical irradiation fiber probe is used, for instance, in photoimmunotherapy, which is one of the treatment methods for cancer. In such a case, the distal end of an optical transmission cable is inserted into the human body to emit laser light onto a drug that has been administered to the body and has reached cancer cells.

### CITATION LIST

### Patent Document

Patent Document 1: Japanese U.S. Pat. No. 4,659,137

### DISCLOSURE OF THE INVENTION

### Problems to Be Solved by the Invention

In photoimmunotherapy, light irradiation should be executed in a state where the distal end of the optical transmission cable is inserted into the human body or positioned near the surface of a tumor. In organs such as the intestines and esophagus, cancer cells as the target of irradiation are often located on the lateral surfaces of the organ. Therefore, it is important to efficiently emit light at an oblique angle relative to the axial direction of the optical transmission cable. According to Patent Document 1, the prism lens with an inclined distal surface allows light to be emitted in a direction inclined relative to the axial direction of the optical transmission cable. However, issues such as beam splitting or the inability to sufficiently expand the beam over the irradiation distance may arise. When an inclined surface or similar processing is applied to the fiber end face to bend light by setting the angle of a single refractive surface, the incidence angle of the refractive surface often approaches the critical angle. As a result, when light with an incidence angle distributed over a certain range enters the refractive surface, both reflected light and transmitted light are generated, ultimately causing the incident beam to split.

The present invention aims to provide an optical irradiation fiber probe capable of emitting high-quality light beams in a direction inclined relative to the axial direction of the optical transmission cable.

### Means for Solving the Problems

(1) The optical irradiation fiber probe is configured to be installed in a therapeutic medical device and includes: an optical transmission cable configured to transmit light emitted from a light source; and a light refraction part provided on a distal end side of the optical transmission cable and configured to refract the light emitted from the optical transmission cable via at least two refractive surfaces, thereby emitting light inclined at least at a predetermined angle relative to an axial direction of the optical transmission cable.

(2) In the optical irradiation fiber probe as described in (1), assuming that the refractive index of the medium with the lower refractive index among the two media partitioned by the refractive surfaces is defined as ns, and the refractive index of the medium with the higher refractive index is defined as n1, the incidence angle θi of the light entering each of the at least two refractive surfaces satisfies the following Equation (1) for at least a part of the entirety of the optical path:

θi≤arcsin(ns/n1). (Equation (1) )

Equation (1) is desirably satisfied for at least a part or the entirety of the optical path.

(3) In the optical irradiation fiber probe as described in (1), at least one of the at least two refractive surfaces is curved.

(4) In the optical irradiation fiber probe as described in any one of (1) to (3), the optical transmission cable includes a core and a cladding formed around an outer periphery of the core, the light refraction part includes a spherical lens, the refractive surfaces are formed by the surface of the lens, and the center of the lens is arranged at a position that does not overlap with the cross-sectional center of the core in the axial view of the core.

(5) The optical irradiation fiber probe as described in (4) is a polymer-clad optical fiber, in which the cladding is made of a hard polymer material.

(6) In the optical irradiation fiber probe as described in (4) or (5), the lens in the light refraction part has an outer diameter larger than an outer diameter of the optical transmission cable, and the center of the lens is arranged at a position that is offset from the cross-sectional center of the core by a distance corresponding to at least ¼ and less than ⅜ of the diameter of the lens in the axial view of the core.

(7) The optical irradiation fiber probe as described in any one of (4) to (6) further includes a retaining part configured to retain the relative position of the distal end of the optical transmission cable relative to the lens without displacement.

(8) The optical irradiation fiber probe as described in any one of (1) to (7) further includes a light shielding part arranged on a side opposite to the emission direction of the light emitted from the light refraction part.

### Effects of the Invention

According to the present invention, high-quality light beams can be emitted in a direction inclined relative to the axial direction of the optical transmission cable.

### BRIEF DESCRIPTION OF THE DRAWINGS

FIG. 1 is a side view illustrating an optical irradiation fiber probe according to a first embodiment of the present invention;

FIG. 2 is a cross-sectional view taken along the line II-II in FIG. 1;

FIG. 3 is a cross-sectional view taken along the line III-III in FIG. 1;

FIG. 4 is a schematic diagram illustrating the relationship between the relative position of the optical transmission cable and the ball lens, and the path of the laser light, in the optical irradiation fiber probe according to the first embodiment of the present invention;

FIG. 5 is a schematic diagram illustrating the relationship between the relative position of an optical transmission cable with a diameter larger than that of the optical transmission cable illustrated in FIG. 4 and the ball lens, and the path of the laser light;

FIG. 6 is a side view illustrating an optical irradiation fiber probe according to a second embodiment of the present invention;

FIG. 7 is a schematic diagram illustrating the relationship between the relative position of the optical transmission cable and the ball lens, and the path of the laser light, in an optical irradiation fiber probe according to a third embodiment of the present invention;

FIG. 8A is a schematic diagram illustrating a conceptual representation of the light intensity distribution of laser light emitted from an optical transmission cable, which is a polymer-clad optical fiber, without passing through a light refraction part;

FIG. 8B is a schematic diagram illustrating a conceptual representation of the light intensity distribution of laser light emitted from an optical transmission cable, which is a plastic optical fiber, via a light refraction part;

FIG. 8C is a schematic diagram illustrating a conceptual representation of the light intensity distribution of laser light emitted from an optical transmission cable, which is a polymer-clad optical fiber, via a light refraction part;

FIG. 9 is a schematic diagram illustrating the relationship between the laser light path and the position in the case where the center of the ball lens is displaced from the cross-sectional center of the core by a distance corresponding to ⅜ of the diameter of the ball lens in the axial view of the core;

FIG. 10 is a schematic diagram illustrating the relationship between the laser light path and the position in the case where the center of the ball lens is displaced from the cross-sectional center of the core by a distance corresponding to ⅙ of the diameter of the ball lens, in the axial view of the core; and

FIG. 11 is a side view illustrating a modification of the refraction part of the optical irradiation fiber probe of the present invention.

### PREFERRED MODE FOR CARRYING OUT THE INVENTION

Hereinafter, the embodiments of the present invention will be described with reference to the drawings. The present invention is not limited to the embodiments described below. The drawings referred to in the following description are merely schematic representations that depict the shapes, sizes, and positional relationships of components to an extent sufficient for understanding the disclosure. Therefore, the present invention is not limited to the shapes, sizes, and positional relationships illustrated in the drawings.

### First Embodiment

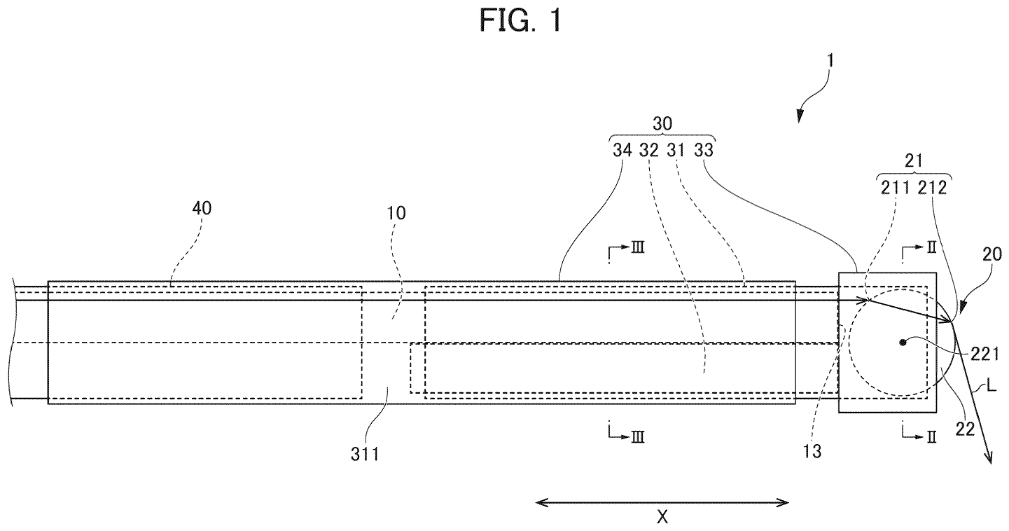

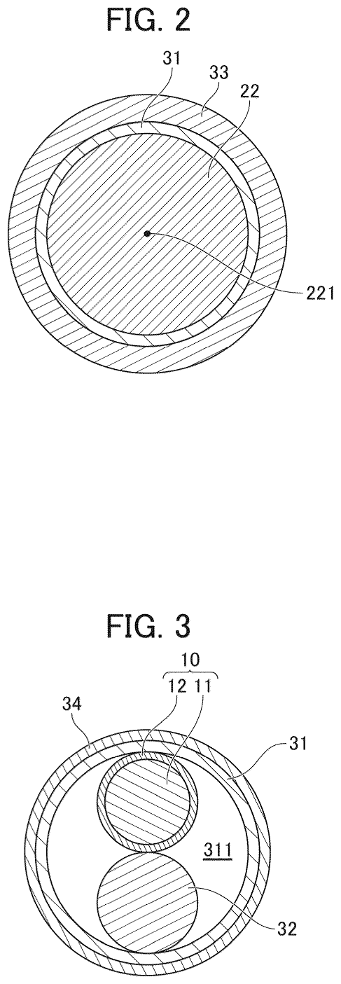

An optical irradiation fiber probe 1 according to a first embodiment of the present invention will be described with reference to FIGS. 1 to 3. FIG. 1 is a side view of the optical irradiation fiber probe 1. FIG. 2 is a cross-sectional view taken along the line II-II in FIG. 1. FIG. 3 is a cross-sectional view taken along the line III-III in FIG. 1.

The optical irradiation fiber probe 1 of the present embodiment is configured to be used in a medical device for photoimmunotherapy, which is one of cancer treatment methods. That is, the optical irradiation fiber probe 1 is installed in a therapeutic medical device. Photoimmunotherapy involves administering a drug including an antibody capable of binding to cancer cells and a photosensitive substance, followed by irradiating the drug bound to the cancer cells with laser light to destroy the cancer cells, thereby treating cancer. The present invention is not limited to photoimmunotherapy and can also be applied to other laser light-based treatment methods, such as photodynamic therapy. The optical irradiation fiber probe 1 is inserted into a lumen provided in an endoscope, and used in a state where the distal end thereof is exposed externally.

As illustrated in FIG. 1, the optical irradiation fiber probe 1 includes an optical transmission cable 10, a light refraction part 20, a retaining part 30, and a cable protective member 40. The optical irradiation fiber probe 1 emits laser light L generated by a laser oscillator (not illustrated) serving as the light source. In the optical irradiation fiber probe 1, a lens restraining member 33 or a cable restraining member 34 (described later) in the retaining part 30 is arranged on the outermost side, while the light refraction part 20, the optical transmission cable 10, and an intermediate member 32 are arranged on the innermost side. Therefore, in FIG. 1, portions exposed to the outside are indicated by solid lines, while portions covered by other members and not exposed to the outside are indicated by dashed lines.

The laser oscillator includes a semiconductor laser, and generates laser oscillation by applying electricity to the semiconductor laser, thereby generating laser light L. The laser oscillator generates red laser light L with a wavelength between 600 nm and 700 nm inclusive. The laser light L generated by the laser oscillator may be a continuous wave or a pulsed wave. The beam mode of the laser light L generated by the laser oscillator may be either a single mode or a multimode. The beam mode of the laser light L is preferably a multimode which allows high-intensity light to be emitted over a wide area. That is, a multimode light source is preferably used.

The optical transmission cable 10 is an optical fiber cable including an optical transmission path for transmitting the laser light L emitted from the laser oscillator. A laser oscillator is arranged at the proximal end of the optical transmission cable 10, while a light refraction part 20 is provided on the distal end 13 side. The optical transmission cable 10 transmits the laser light L generated by the laser oscillator through the optical transmission path, and emits the laser light L toward the light refraction part 20 from the distal end 13.

The optical transmission cable 10 of the present embodiment is a plastic optical fiber, and includes a core 11 serving as an optical transmission path, and a cladding 12 formed around the outer periphery of the core 11. The optical transmission cable 10 has, for example, an outer diameter of 500 μm, while the core 11 has an outer diameter of 250 μm. The core 11 preferably has an outer diameter suitable for multimode fibers. The optical transmission cable 10 in the present embodiment is a single-core optical fiber, and may also be a multi-core optical fiber. Furthermore, the shape of the core may be a perfect circle, elliptical, or rectangular. The optical transmission cable 10 may be an optical fiber made of a silica-based material. In a case where the optical transmission cable 10 is a plastic optical fiber, such an optical transmission cable 10 can be bent more easily.

The light refraction part 20 is arranged on the distal end 13 side of the optical transmission cable 10, and emits the laser light L, which is emitted from the core 11 at the distal end 13 of the optical transmission cable 10, at an angle inclined by at least at a predetermined angle relative to the axial direction X of the optical transmission cable 10. The axial direction X of the optical transmission cable 10 in the present specification refers to the axial direction of the optical transmission cable 10 at the distal end 13.

The light refraction part 20 is provided with at least two curved refractive surfaces 21 configured to refract the laser light L emitted from the optical transmission cable 10. The light refraction part 20 of the present embodiment includes, for example, a ball lens 22 being a spherical lens. The ball lens 22 has, for example, a refractive index of 1.5 and a diameter of 1 mm. The refractive surface 21 of the light refraction part 20 is formed by the surface of the ball lens 22. Specifically, the refractive surface 21 of the light refraction part 20 includes an incident surface 211, on which the light emitted from the distal end 13 of the optical transmission cable 10 is incident, and an emission surface 212, from which the incident light is emitted. The incident surface 211 is formed as a curved surface protruding toward the optical transmission cable 10. The emission surface 212 is formed as a curved surface protruding in the direction of light emission. As illustrated in FIG. 1, the light refraction part 20 refracts the laser light L emitted from the distal end 13 of the optical transmission cable 10 at the incident surface 211, causing the light to enter the ball lens 22. The light refraction part 20 then refracts the laser light L within the ball lens 22 at the emission surface 212, causing the light to be emitted outside the ball lens 22. That is, the light refraction part 20 refracts the laser light L emitted from the optical transmission cable 10 through at least two refractive surfaces 21, and emits the laser light L at an angle inclined by at least at a predetermined angle relative to the axial direction X of the optical transmission cable 10. The predetermined angle may be, for example, at least 20 degrees or more. In the present embodiment, all refractive surfaces 21 of the light refraction part 20 are curved. However, at least one of the two or more refractive surfaces 21 may be curved, or all of the refractive surfaces 21 may not be curved. For example, the refractive surface 21 may be a flat surface.

The ball lens 22 of the light refraction part 20 is arranged at a predetermined position relative to the optical transmission cable 10. Specifically, the center 221 of the ball lens 22 is arranged at a position that does not overlap with the core 11 in the axial view of the distal end 13. That is, the center 221 of the ball lens 22 is arranged at a position that is offset from the cross-sectional center of the core 11 when viewed in the axial direction X.

The retaining part 30 retains the relative position of the distal end 13 of the optical transmission cable 10 relative to the ball lens 22 of the light refraction part 20 without displacement. The retaining part 30 includes a tubular member 31, an intermediate member 32, a lens restraining member 33, and a cable restraining member 34.

The tubular member 31 is a flexible cylindrical tube. A cavity 311 inside the tubular member 31 accommodates at least part of the optical transmission cable 10 and part of the ball lens 22. Specifically, the optical transmission cable 10 is inserted into the tubular member 31 such that at least the distal end 13 side is positioned within the cavity 311 of the tubular member 31. As illustrated in FIG. 1, the optical transmission cable 10 is accommodated in the cavity 311 of the tubular member 31 in a state of extending in the axial direction of the tubular member 31. The ball lens 22 is accommodated in the tubular member 31 such that at least the incident surface 211 side is positioned within the cavity 311 of the tubular member 31, while the emission surface 212 side is exposed outside the tubular member 31. The material of the tubular member 31 may include, for example, polyimide, silicone, or polytetrafluoroethylene (PTFE).

The intermediate member 32 is a flexible elongated member accommodated in the cavity 311 of the tubular member 31. The intermediate member 32 is arranged on the distal end 13 side along the optical transmission cable 10 inside the tubular member 31, filling the gap between the outer peripheral surface of the optical transmission cable 10 and the inner peripheral surface of the tubular member 31. As illustrated in FIG. 3, the shape of the intermediate member 32 is cylindrical; however, the shape thereof is not particularly limited. For example, the shape of the intermediate member 32 may be tubular or plate-shaped.

The lens restraining member 33 is a member configured to prevent movement of the ball lens 22 within the tubular member 31. In the present embodiment, the lens restraining member 33 is, for example, a cylindrical heat-shrinkable tube capable of reducing in diameter upon heating. As illustrated in FIGS. 1 and 2, the lens restraining member 33 covers the portion accommodating the ball lens 22 in the tubular member 31. That is, as illustrated in FIG. 2, the ball lens 22 is positioned at the innermost side of the optical irradiation fiber probe 1, the lens restraining member 33 is positioned at the outermost side, and the tubular member 31 is positioned between the ball lens 22 and the lens restraining member 33. In this state, the lens restraining member 33 is heat-shrunk, applying a force directed toward the inner diameter of the ball lens 22. As a result, the tubular member 31 and the ball lens 22 become tightly adhered to each other.

The cable restraining member 34 is a member configured to restrain the optical transmission cable 10 and the intermediate member 32 so as not to move within the tubular member 31. In the present embodiment, the cable restraining member 34 is, for example, a cylindrical heat-shrinkable tube capable of reducing in diameter upon heating. As illustrated in FIGS. 1 and 3, the cable restraining member 34 covers at least part of the portion accommodating the optical transmission cable 10 and the intermediate member 32 in the tubular member 31, as well as part of the cable protective member 40. As illustrated in FIG. 3, the optical transmission cable 10 and the intermediate member 32 are positioned within the cavity 311 of the tubular member 31, and the cable restraining member 34 is positioned outside the tubular member 31. In this state, the cable restraining member 34 is heat-shrunk, applying a force directed toward the inner diameter of the tubular member 31. As a result, the optical transmission cable 10, the intermediate member 32, and the tubular member 31 become tightly adhered to each other. The lens restraining member 33 and the cable restraining member 34, both of which are heat-shrinkable tubes, are heat-shrunk during the manufacturing process of the optical irradiation fiber probe 1. The material of the heat-shrinkable tubes may include, for example, FEP (a fluororesin that is a copolymer of tetrafluoroethylene and hexafluoropropylene).

The retaining part 30 accommodates at least the distal end 13 side of the optical transmission cable 10 including the distal end 13, and at least the distal end 13 side of the ball lens 22 within the tubular member 31. The position of the distal end 13 of the optical transmission cable 10 in the tubular member 31 is fixed by the intermediate member 32 and the cable restraining member 34, while the position of the ball lens 22 in the tubular member 31 is fixed by the lens restraining member 33. That is, the retaining part 30 retains the relative position of the optical transmission cable 10 relative to the ball lens 22.

The cable protective member 40 is a flexible cylindrical tube configured to protect the optical transmission cable 10. As illustrated in FIG. 1, the cavity inside the cable protective member 40 accommodates a portion of the optical transmission cable 10 that is not inserted into the tubular member 31, including the proximal end side of the optical transmission cable 10. The cable protective member 40 is arranged in the extending direction of the optical transmission cable 10, with a gap between the cable protective member 40 and the optical transmission cable 10. The material of the cable protective member 40 may include, for example, nylon, silicone, or polytetrafluoroethylene (PTFE).

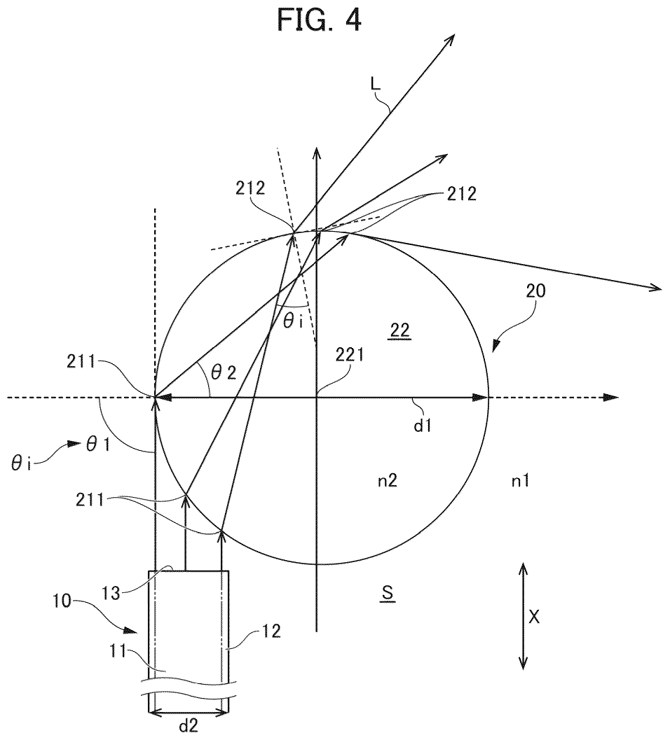

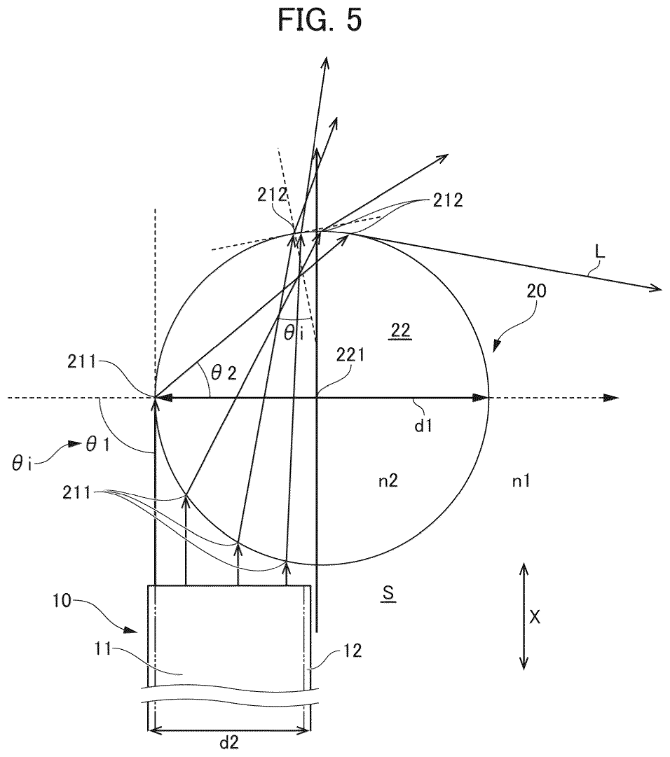

Next, the relationship between the relative position of the optical transmission cable 10 and the light refraction part 20, and the path of the laser light L, will be described with reference to FIGS. 4 and 5. FIG. 4 is a schematic diagram illustrating the relationship between the relative position of the optical transmission cable 10 and the ball lens 22, and the path of the laser light L. FIG. 5 is a schematic diagram illustrating the relationship between the relative position of an optical transmission cable with a diameter larger than that of the optical transmission cable illustrated in FIG. 4 and the ball lens 22, and the path of the laser light L. The optical transmission cable 10 illustrated in FIG. 4 has a diameter of 250 μm, while the optical transmission cable 10 illustrated in FIG. 5 has a diameter of 500 μm. The ball lens 22 illustrated in FIGS. 4 and 5 has a diameter of 1000 μm and a refractive index n2 of 1.5. The refractive index n1 of the space S surrounding the ball lens 22 and the optical transmission cable 10, as illustrated in FIGS. 4 and 5, is 1.0.

The laser light L emitted from the optical transmission cable 10 is refracted at an angle derived from Snell's law described below in Equation (0), at the incident surface 211 of the ball lens 22. The light then passes through the ball lens 22, is refracted again at the emission surface 212, which is the boundary between the ball lens 22 and the surrounding space S, and is emitted outward.

sin θ1/sin θ2=n2/n1. (Equation (0) )

Here, “θ1” represents the incident angle of the light entering the ball lens 22 at the incident surface 211 from the space S. “θ2” represents the refraction angle of the laser light L at the incident surface 211.

Since the laser light L is emitted from the distal end 13 of the optical transmission cable 10 onto the ball lens 22 being a sphere, the path of the laser light L varies depending on the entry position of the laser light L into the ball lens 22. Specifically, as illustrated in FIGS. 4 and 5, in the axial view of the core 11, the farther the incident surface 211 is from the center 221 of the ball lens 22, the larger the angle at which the laser light L is inclined relative to the axial direction X. The laser light L having passed through the ball lens 22 is then refracted again at the emission surface 212 and emitted at an even larger angle relative to the axial direction X. Accordingly, the laser light L can also be emitted toward a wider lateral area from the optical irradiation fiber probe 1. Since the laser light L changes the traveling direction over a plurality of times rather than abruptly at a single point, beam splitting due to refraction of the laser light L can be suppressed. Conversely, as illustrated in FIG. 5, the closer the incident surface 211 is to the center 221 in the axial view of the core 11, the smaller the change in the traveling direction of the laser light L. In other words, the path of the laser light L emitted from the distal end 13 of the optical transmission cable 10 becomes more linear. In the present embodiment, in order to efficiently irradiate the target cells located more laterally rather than in the front of the optical irradiation fiber probe 1, the center 221 of the ball lens 22 is arranged at a position that is offset from the cross-sectional center of the core 11 in the axial view of the core 11.

Assuming that the refractive index of the medium with the lower refractive index among the two media partitioned by the refractive surface 21 is defined as ns, and the refractive index of the medium with the higher refractive index is defined as n1, the incident angle θi of the light incident on each of the at least two refractive surfaces 21 preferably satisfies the following Equation (1) for at least a part or the entirety of the optical path. With this configuration, the incidence angle θi, for example, becomes equal to or lower than the critical angle, thereby suppressing beam splitting in the incident beam.

θi≤arcsin(ns/n1). (Equation (1) )

The right-hand side of Equation (1) corresponds to the critical angle. When light enters from a higher refractive index medium into a lower refractive index medium, total internal reflection occurs at this angle. However, even in the reverse case (where light enters from a lower refractive index medium into a higher refractive index medium), high reflection occurs, leading to beam splitting. It is more preferable that Equation (1) is satisfied for the entire optical path.

In the example illustrated in FIGS. 4 and 5, since the refractive index of the ball lens 22 is greater than that of the space S, the incidence angle θi of the light including θ1 preferably satisfies the following Equation (2):

θi≤arcsin(n1/n2). (Equation (2) )

As illustrated in FIGS. 4 and 5, the emission range of the light emitted from the light refraction part 20 can be expanded by adjusting the diameter of the optical transmission cable 10 relative to the ball lens 22. This is the characteristic and effect of using a spherical refractive surface such as a ball lens. In this manner, the traveling direction and emission range of the laser light L can be controlled by adjusting the relative position of the optical transmission cable 10 and the ball lens 22, as well as the diameter of the core 11 of the optical transmission cable 10 relative to the ball lens 22.

### Second Embodiment

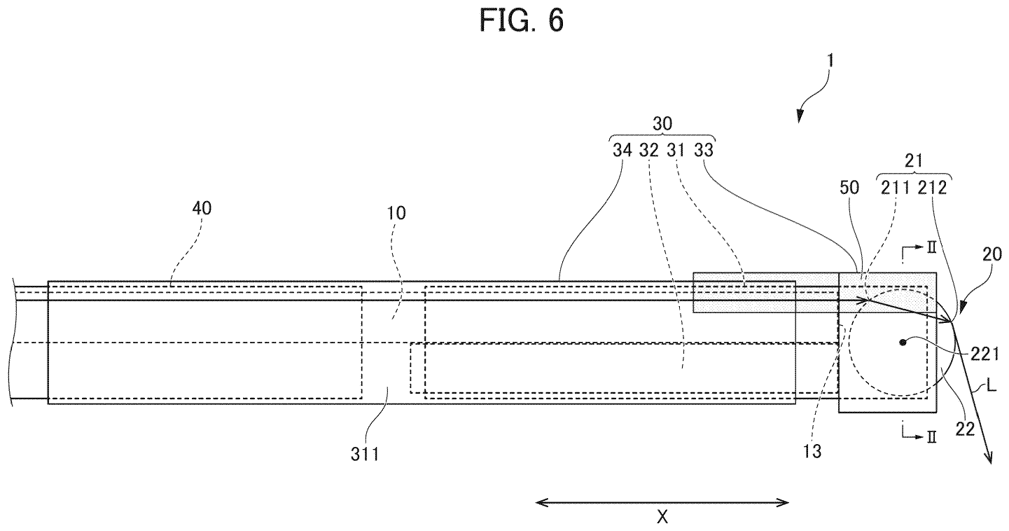

Next, an optical irradiation fiber probe 1 according to a second embodiment of the present invention will be described with reference to FIG. 6. FIG. 6 is a side view illustrating the optical irradiation fiber probe 1 according to the second embodiment. In the following description of the second embodiment, components corresponding to those in the first embodiment are denoted by corresponding reference numerals, based on the same rules. Descriptions may be omitted or incorporated by reference as needed.

The optical irradiation fiber probe 1 of the present embodiment includes the optical transmission cable 10, the light refraction part 20, the retaining part 30, the cable protective member 40, and a light shielding part 50. The primary difference from the optical irradiation fiber probe 1 of the first embodiment is that the optical irradiation fiber probe 1 of the present embodiment includes the light shielding part 50.

The light shielding part 50 is arranged on the side opposite to the emission direction of the laser light L emitted from the light refraction part 20. Specifically, the light shielding part 50 is formed by applying a light-shielding material to the side opposite to the emission direction of the laser light L emitted from the light refraction part 20 in the retaining part 30. In the example illustrated in FIG. 6, the light-shielding material is applied to the surfaces of the lens restraining member 33, the portion of the tubular member 31 on the ball lens 22 side, and the cable restraining member 34 on the side opposite to the emission direction of the laser light L (the upper side in FIG. 6). The light-shielding material may include, for example, metals or polytetrafluoroethylene (PTFE).

### Third Embodiment

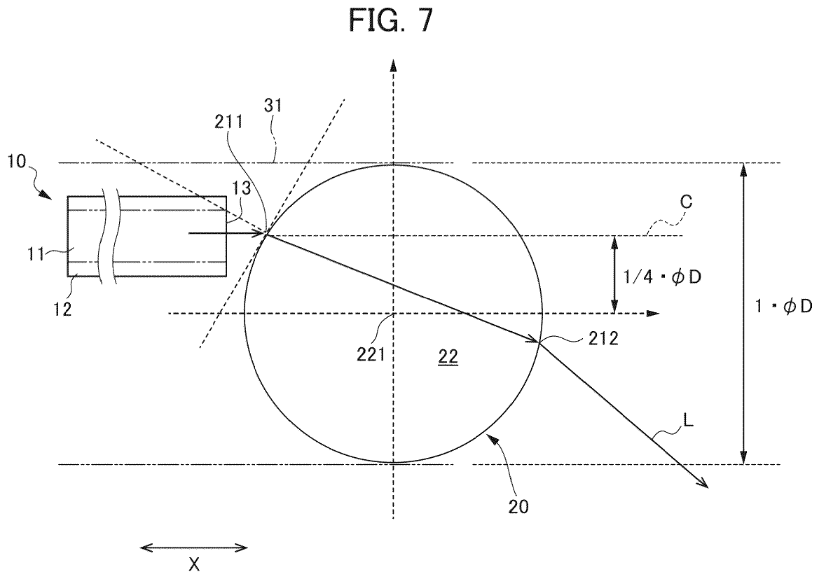

Next, an optical irradiation fiber probe 1 according to a third embodiment of the present invention will be described with reference to FIG. 7. In the following description of the third embodiment, components corresponding to those in the first embodiment are denoted by corresponding reference numerals, based on the same rules. Descriptions may be omitted or incorporated by reference as needed. FIG. 7 is a schematic diagram illustrating the relationship between the relative position of the optical transmission cable 10 and the ball lens 22, and the path of the laser light L in the optical irradiation fiber probe 1 of the third embodiment. In FIG. 7, illustrations of components other than the optical transmission cable 10, the light refraction part 20, and the tubular member 31 are omitted. In FIG. 7, the path of the component at the center of the light intensity distribution of the laser light L is illustrated, in which the dashed line C represents a virtual line extending along the axial direction X of the optical transmission cable 10 from the cross-sectional center of the core 11 toward the ball lens 22.

The optical irradiation fiber probe 1 of the present embodiment includes the optical transmission cable 10, the light refraction part 20, the retaining part 30, and the cable protective member 40. The primary difference from the optical irradiation fiber probe 1 of the first embodiment is the configuration of the optical transmission cable 10 of the optical irradiation fiber probe 1 of the present embodiment.

The optical transmission cable 10 of the present embodiment is a polymer-clad optical fiber, in which the cladding 12 is made of a hard polymer material. A polymer-clad optical fiber refers to an optical fiber, in which the core 11 is formed of a silica-based material and the cladding 12 is formed of a hard polymer material. Examples of the silica-based material include, for example, silica with no impurities doped into the core, or germanium-doped silica. Examples of the hard polymer material may include, for example, fluororesins such as PTFE, PVDF, and ETFE, polyimide, or copolymers thereof. The optical transmission cable 10 of the present embodiment has an outer diameter smaller than the outer diameter of the ball lens 22 in the light refraction part 20.

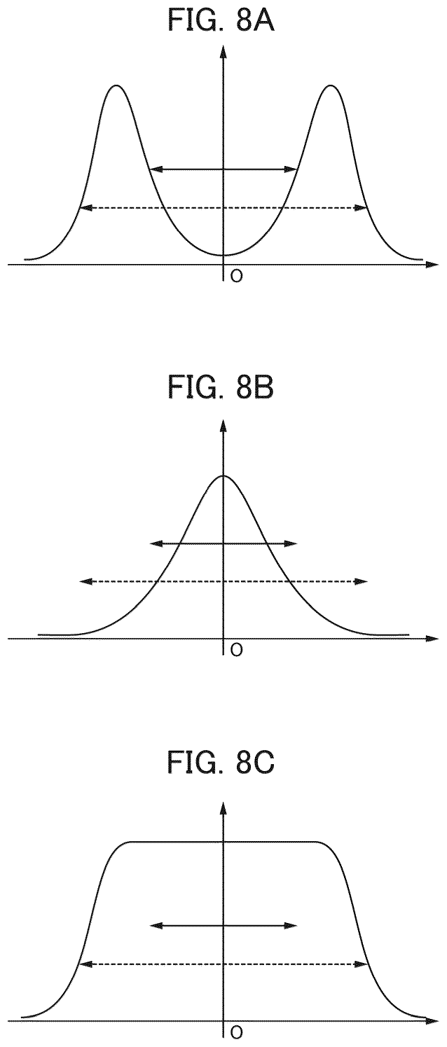

Here, the light intensity distribution of the laser light L emitted from the optical irradiation fiber probe 1 of the third embodiment will be described with reference to FIGS. 8A to 8C. FIGS. 8A to 8C are explanatory diagrams illustrating the light intensity distribution of the laser light L emitted from the optical irradiation fiber probe 1 of the third embodiment. FIG. 8A is a schematic diagram illustrating a conceptual representation of the light intensity distribution of the laser light L emitted from the distal end 13 of the optical transmission cable 10 of the third embodiment, which is a polymer-clad optical fiber, without passing through the light refraction part 20. FIG. 8B is a schematic diagram illustrating a conceptual representation of the light intensity distribution of the laser light L emitted from the distal end 13 of the optical transmission cable 10 of the first embodiment, which is a plastic optical fiber, via the light refraction part 20. FIG. 8C is a schematic diagram illustrating a conceptual representation of the light intensity distribution of the laser light L emitted from the optical irradiation fiber probe 1 of the third embodiment via the light refraction part 20. In FIGS. 8A to 8C, the vertical axis represents light intensity, and the horizontal axis represents the cross-sectional distance from the center of the laser light L, assuming that the origin 0 is the center of the laser light L in the cross-section of the laser light L. That is, in FIGS. 8A to 8C, the region around the origin O on the horizontal axis represents the area where the core 11 is located on the optical transmission cable 10 side along the optical axis direction of the laser light L. In FIGS. 8A to 8C, the range indicated by the solid double-headed arrow on the horizontal axis represents the region where the core is present in the optical axis direction of the laser light L on the optical transmission cable side, while the range indicated by the dashed double-headed arrow represents the region where both the core and the cladding are present in the optical axis direction of the laser light L on the optical transmission cable side.

As illustrated in FIG. 8A, the laser light L emitted from the distal end 13 of the optical transmission cable 10, which is a polymer-clad optical fiber, has the light intensity distribution concentrated in the region where the cladding exists (hereinafter referred to as the “cladding region”) in the optical axis direction of the laser light L on the optical transmission cable 10 side. This is because the polymer-clad optical fiber allows the refractive index of the cladding to be controlled by adjusting the composition 4 the cladding resin. For example, the laser light can be concentrated in the cladding region by adjusting the relative refractive index difference to Δ=3 to 5% (NA=0.37 to 0.47).

On the other hand, as illustrated in FIG. 8B, the laser light L emitted from the distal end 13 of an optical transmission cable 10 that is not a polymer-clad optical fiber, such as a plastic fiber, via the light refraction part 20 exhibits a Gaussian-like intensity distribution, with the light intensity concentrated at the center of the core 11 in the optical axis direction of the laser light L on the optical transmission cable 10 side. This is because the laser light L is focused by the light refraction part 20, such as the ball lens 22.

The laser light L emitted from the distal end 13 of the optical transmission cable 10 being a polymer-clad optical fiber via the ball lens 22 has the light intensity distribution, which is concentrated in the cladding region, passing through the ball lens 22 focusing the light. As a result, the light intensity distributions illustrated in FIG. 8A and FIG. 8B offset each other, forming a flat-top light intensity distribution as illustrated in FIG. 8C. A flat-top light intensity distribution refers to a light intensity distribution in which the variation in light intensity within a predetermined radius from the center of the laser light L is minimal and uniform, and the light intensity rapidly decreases beyond that predetermined radius. Accordingly, even in a case where the laser light L is emitted in a direction inclined relative to the insertion direction of the optical transmission cable 10, the laser light L can be emitted with stronger light intensity even onto lateral surfaces further away from the optical transmission cable 10. Therefore, the laser light L with high treatment efficiency can irradiate the irradiation target such as cancer cells existing on the lateral surfaces of an organ.

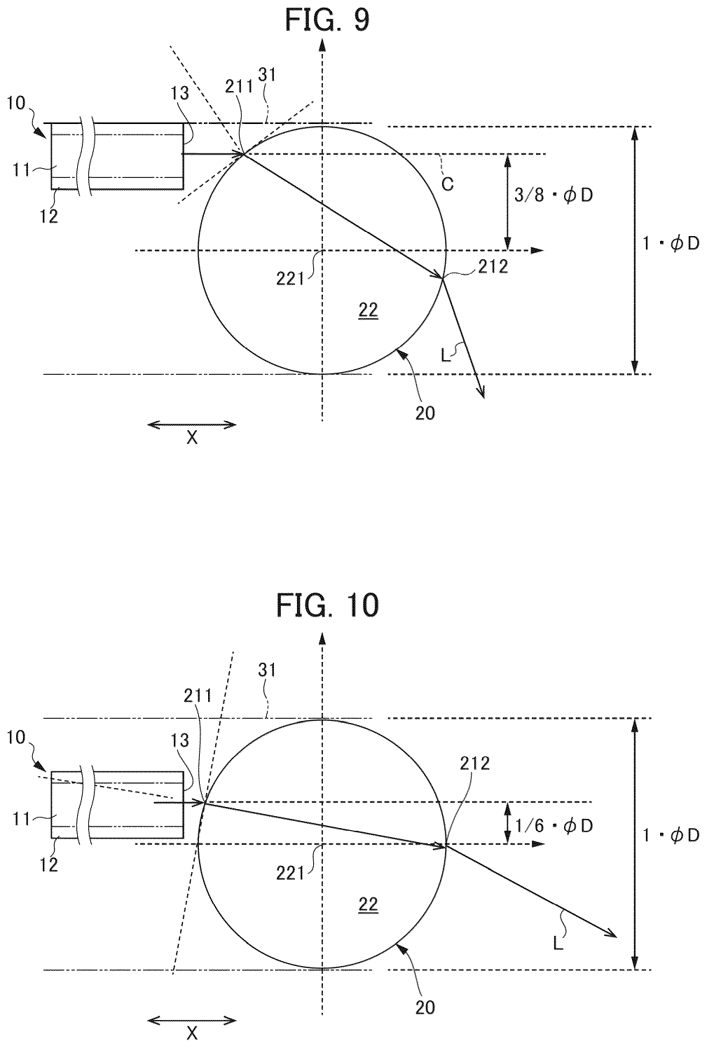

Next, the relationship between the relative position of the optical transmission cable 10 and the ball lens 22 in the optical irradiation fiber probe 1, and the path of the laser light L, will be described with reference to FIGS. 7, 9, and 10. FIG. 7 is a schematic diagram illustrating the relationship between the position, in which the center 221 of the ball lens 22 is offset from the cross-sectional center of the core 11 by a distance corresponding to ¼ of the diameter of the ball lens 22 in the axial view of the core 11, and the path of the laser light L. FIG. 9 is a schematic diagram illustrating the relationship between the position, in which the center 221 of the ball lens 22 is offset from the cross-sectional center of the core 11 by a distance corresponding to ⅜ of the diameter of the ball lens 22 in the axial view of the core 11, and the path of the laser light L. FIG. 10 is a schematic diagram illustrating the relationship between the position, in which the center 221 of the ball lens 22 is offset from the cross-sectional center of the core 11 by a distance corresponding to ⅙ of the diameter of the ball lens 22 in the axial view of the core 11, and the path of the laser light L. FIGS. 9 and 10 omit illustrations of components other than the optical transmission cable 10, the light refraction part 20, and the tubular member 31. FIGS. 9 and 10 illustrate the path of the component at the center of the light intensity distribution of the laser light L, in which the dashed line C represents a virtual line extending along the axial direction X of the optical transmission cable 10 from the cross-sectional center of the core 11 toward the ball lens 22.

The ball lens 22 of the light refraction part 20 in the present embodiment has an outer diameter larger than the outer diameter of the optical transmission cable 10. The center 221 of the ball lens 22 is preferably arranged at a position that is offset from the cross-sectional center of the core 11 by a distance corresponding to at least ¼ (at least ¼φ(D) but less than ⅜ (less than ⅜φ(D) of the diameter of the ball lens 22 in the axial view of the core 11.

As illustrated in FIG. 7, in a case where the center 221 of the ball lens 22 is offset from the cross-sectional center of the core 11 by a distance corresponding to ¼φD in the axial view of the core 11, the path of the component at the center of the light intensity distribution allows the laser light L to be emitted at an angle inclined by 45° relative to the insertion direction of the optical transmission cable 10.

As illustrated in FIG. 9, in a case where the center 221 of the ball lens 22 is offset from the cross-sectional center of the core 11 by a distance corresponding to ⅜φD of the ball lens 22 from the cross-sectional center of the core 11 in the axial view of the core 11, the laser light L can be emitted in a direction, in which the path of the component at the center of the light intensity distribution is further inclined relative to the insertion direction of the optical transmission cable 10, as compared to the case in FIG. 7. However, a tubular member 31 accommodating part of the optical transmission cable 10 or part of the ball lens 22 exists outside the optical transmission cable 10. Therefore, considering potential interference with the tubular member 31, the size of the optical transmission cable 10 should be reduced with the increase of the offset distance of the core 11 relative to the center 221 of the ball lens 22. From the perspective of emitting the laser light L over a wider area from the optical transmission cable 10, the center 221 of the ball lens 22 is preferably arranged at a position that is offset from the cross-sectional center of the core 11 by a distance corresponding to less than ⅜φD of the ball lens 22 in the axial view of the core 11.

Conversely, in a case where the distance between the center 221 of the ball lens 22 and the cross-sectional center of the core 11 in the axial view of the core 11 is set to be less than ¼φD of the ball lens 22, as illustrated in FIG. 10, the angle at which the laser light L is emitted relative to the insertion direction of the optical transmission cable 10 becomes shallower. Therefore, in order to efficiently irradiate the irradiation target such as cancer cells existing on the lateral surfaces of an organ with the laser light L, the distance between the center 221 of the ball lens 22 and the cross-sectional center of the core 11 is preferably set to be at least ¼φD of the ball lens 22.

According to the embodiments described above, the following effects can be achieved.

The optical irradiation fiber probe 1 according to the present embodiment is installed in a therapeutic medical device and includes: an optical transmission cable 10 configured to transmit light emitted from a light source; and a light refraction part 20, which is provided on the distal end 13 side of the optical transmission cable 10 and refracts the laser light L emitted from the optical transmission cable 10 via at least two refractive surfaces 21, thereby emitting light inclined at least at a predetermined angle relative to the axial direction X of the optical transmission cable 10.

Accordingly, since the light is refracted a plurality of times through at least two refractive surfaces 21 before being emitted, heat generation during the emission of the laser light L can be suppressed, while minimizing beam splitting and enabling the emission of high-quality laser light L in a direction inclined relative to the insertion direction of the optical transmission cable 10. Since the emission range of the laser light L can be expanded over a short irradiation distance, the laser light L can efficiently irradiate cancer cells or other targets located on the surface of narrow and elongated organs within the human body.

In the optical irradiation fiber probe 1 according to the present embodiment, assuming that the refractive index of the medium with the lower refractive index among the two media partitioned by the refractive surface 21 is defined as ns, and the refractive index of the medium with the higher refractive index is defined as n1, the incident angle θi of the light incident on each of the at least two refractive surfaces 21 satisfies the following Equation (1) for at least a part or the entirety of the optical path:

θi≤arcsin(ns/n1). (Equation (1) )

Accordingly, beam splitting can be further suppressed, and laser light L with even higher beam quality can be emitted.

In the optical irradiation fiber probe 1 of the present embodiment, at least one of the at least two refractive surfaces 21 is curved.

Accordingly, since the laser light L is refracted a plurality of times through at least one curved refractive surface 21 and then emitted, the emission angle of the laser light L relative to the insertion direction of the optical transmission cable 10 can be increased.

In the optical irradiation fiber probe 1 of the present embodiment, the optical transmission cable 10 includes the core 11 and the cladding 12 formed around the outer periphery of the core 11, the light refraction part 20 includes the spherical ball lens 22, the refractive surface 21 is formed by the surface of the ball lens 22, and the center 221 of the ball lens 22 is arranged at a position that is offset from the cross-sectional center of the core 11 in the axial view of the

Accordingly, since the surface of the ball lens 22 is utilized as the refractive surface 21, the laser light L can be refracted efficiently with a simpler configuration, allowing the laser light L to be emitted in a direction inclined relative to the insertion direction of the optical transmission cable 10 within the human body, even at a short irradiation distance.

In the optical irradiation fiber probe 1 of the present embodiment, the optical transmission cable 10 is a polymer-clad optical fiber, in which the cladding 12 is made of a hard polymer material.

Accordingly, the laser light L emitted from the optical transmission cable 10 and concentrated in the cladding region is emitted externally via the ball lens 22, which functions as a light-focusing element. As a result, the laser light L can be emitted with a flat-top intensity distribution at an inclined angle relative to the insertion direction of the optical transmission cable 10. Consequently, the laser light L with high treatment efficiency can irradiate the irradiation target such as cancer cells existing on the lateral surface of an organ.

In the optical irradiation fiber probe 1 of the present embodiment, the ball lens 22 of the light refraction part 20 has an outer diameter larger than that of the optical transmission cable 10, and the center thereof is arranged at a position that is offset from the cross-sectional center of the core 11 by a distance corresponding to at least ¼ and less than ⅜ of the diameter of the ball lens 22 in the axial view of the core 11.

Accordingly, the laser light L with higher light intensity can be emitted, including the components of the laser light L in a direction inclined at an angle of at least 45° relative to the insertion direction of the optical transmission cable 10.

The optical irradiation fiber probe 1 of the present embodiment includes the retaining part 30 configured to retain the relative position of the distal end 13 of the optical transmission cable 10 relative to the ball lens 22 without displacement.

Accordingly, variations in the emission direction and emission range of the laser light L due to usage can be suppressed.

The optical irradiation fiber probe 1 of the present embodiment includes the light shielding part 50 arranged on the side opposite to the emission direction of the light emitted from the light refraction part 20.

Accordingly, in photoimmunotherapy and photodynamic therapy, which irradiate specific cells such as cancer cells with the laser light L, the irradiation of healthy cells with the laser light L can be suppressed, thereby reducing the invasiveness to the patient body undergoing treatment.

Although the embodiments of the present invention have been described above, the present invention is not limited to the above-described embodiments and can be modified as appropriate.

In the first embodiment described above, the retaining part 30 includes the tubular member 31, the intermediate member 32, the lens restraining member 33, and the cable restraining member 34. However, the configuration may include only the tubular member 31 and the intermediate member 32, without including the lens restraining member 33 and the cable restraining member 34.

In the embodiments described above, the light refraction part 20 includes one ball lens 22 and two refractive surfaces 21. However, the configuration may include a plurality of ball lenses 22. Specifically, the laser light L emitted from the distal end 13 of the optical transmission cable 10 may be refracted by the surfaces of the plurality of ball lenses 22 and then emitted from the optical irradiation fiber probe 1. The sizes and refractive indices of the plurality of ball lenses may be different or may be identical.

In the embodiments described above, the light refraction part 20 is configured to include the ball lens 22, but the configuration of the light refraction part 20 is not particularly limited.

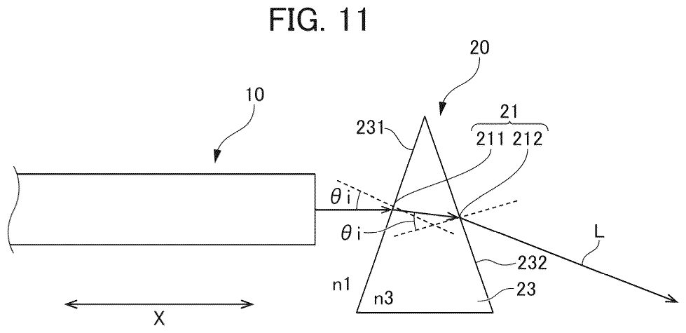

FIG. 11 is a side view illustrating an optical irradiation fiber probe 1 including a light refraction part 20 different from those in the above-described embodiments. FIG. 11 omits illustrations of components other than the optical transmission cable 10 and the light refraction part 20.

The light refraction part 20 illustrated in FIG. 11 includes a triangular prism lens 23 instead of the ball lens 22. The refractive surface 21 of the light refraction part 20 illustrated in FIG. 11 is formed by planar inclined surfaces 231 and 232 of the prism lens 23. In the example illustrated in FIG. 11, assuming that the refractive index of the surrounding space S is defined as n1, and the refractive index of the prism lens 23 is defined as n3, the incidence angle θi of the light satisfies the following Equation (3):

θi≤arcsin(n1/n3). (Equation (3) )

For example, the light refraction part 20 may include a concave lens, which is provided on the distal end 13 side of the optical transmission cable 10. The incident surface, where the light emitted from the distal end 13 of the optical transmission cable 10 enters, may be formed as a curved surface protruding in the light emission direction, while the emission surface, where the light exits, may be formed as a curved surface protruding toward the optical transmission cable 10.

The optical irradiation fiber probe 1 is used as a disposable product in the medical field due to hygiene considerations. However, by recovering the product and reusing components such as lenses, it is possible to contribute to ensuring sustainable consumption and production patterns.

### EXPLANATION OF REFERENCE NUMERALS

1: optical irradiation fiber probe
10: optical transmission cable
13: distal end
20: light refraction part
21: refractive surface
211: incident surface
212: emission surface
X: axial direction of optical transmission cable

1. An optical irradiation fiber probe configured to be installed in a therapeutic medical device, the probe comprising:
an optical transmission cable configured to transmit light emitted from a light source; and
a light refraction part provided on a distal end side of the optical transmission cable and configured to refract the light emitted from the optical transmission cable via at least two refractive surfaces, thereby emitting light inclined at least at a predetermined angle relative to an axial direction of the optical transmission cable.

2. The optical irradiation fiber probe according to claim 1, wherein assuming that a refractive index of a medium with a lower refractive index among two media partitioned by the refractive surfaces is defined as ns, and a refractive index of a medium with a higher refractive index is defined as n1, an incidence angle θi of light entering each of the at least two refractive surfaces satisfies the following Equation (1) for at least a part or the entirety of the optical path:
θi≤arcsin(ns/n1). (Equation (1))

3. The optical irradiation fiber probe according to claim 1, wherein at least one of the at least two refractive surfaces is curved.

4. The optical irradiation fiber probe according to claim 1, wherein
the optical transmission cable includes a core and a cladding formed around an outer periphery of the core,
the light refraction part includes a spherical lens,
the refractive surfaces are formed by a surface of the lens, and
a center of the lens is arranged at a position that is offset from a cross-sectional center of the core in an axial view of the core.

5. The optical irradiation fiber probe according to claim 4, wherein the optical transmission cable is a polymer-clad optical fiber, in which the cladding is made of a hard polymer material.

6. The optical irradiation fiber probe according to claim 4, wherein the lens in the light refraction part has an outer diameter larger than an outer diameter of the optical transmission cable, and the center of the lens is arranged at a position that is offset from the cross-sectional center of the core by a distance corresponding to at least ¼ and less than ⅜ of the diameter of the lens in the axial view of the core.

7. The optical irradiation fiber probe according to claim 4, further comprising a retaining part configured to retain a relative position of the distal end of the optical transmission cable relative to the lens without displacement.

8. The optical irradiation fiber probe according to claim 1, further comprising a light shielding part arranged on a side opposite to an emission direction of the light emitted from the light refraction part.
